(19) United States
(12) Patent Application Publication
(10) Pub. No.: US 2022/0067105 A1
(43) Pub. Date: Mar. 3, 2022

(54) SEARCH ENGINE FOR CONCATENATING AND SEARCHING COMBINATIONS OF DATA FILES

(71) Applicant: University of Pittsburgh - Of the Commonwealth System of Higher Education, Pittsburgh, PA (US)

(72) Inventors: Yoram Vodovotz, Sewickley, PA (US); Fayten El-Dehaibi, Pittsburgh, PA (US); Qi Mi, Pittsburgh, PA (US)

(21) Appl. No.: 17/003,661

(22) Filed: Aug. 26, 2020

(51) Int. Cl. G06F 16/903; G16H 10/60; G16B 50/30; G16B 20/20

(52) U.S. Cl. CPC G06F 16/90344; G16B 20/20; G16B 50/30; G16H 10/60

(57) ABSTRACT

This document describes a search engine that accepts as input different types of data files and conditions for search parameters, including both single and multiple time points, concatenates these data, and outputs data from the different types of files that satisfies the specified search conditions. In one aspect, a method includes receiving a selection of a multiple input data files that each include data on which a search is to be performed. The input data files include different types of data files having different data formats. An in-memory data structure that includes the data of the input data files arranged in a common format is generated. For each of one or more search parameters, data indicating a condition for the search parameter is received. A set of data that satisfies the condition of each of the one or more search parameters is identified in the in-memory data structure.

### STATEMENT OF FEDERALLY SPONSORED RESEARCH

This invention was made with government support under grant number GM053789 awarded by the National Institutes of Health. The government has certain rights in the invention.

### TECHNICAL FIELD

This specification relates to a search engine that accepts as input different types of data files and conditions for search parameters, and outputs data from the different types of files that satisfies the conditions. In some embodiments, the search engine enables searches of genome data, e.g., to extract information about subjects having specified single nucleotide polymorphisms (SNPs) and other genomic or non-genomic conditions.

### BACKGROUND

Data-driven modeling and machine learning analyses have leveraged large datasets to define novel characteristics and putative biological mechanisms in the context of basic biomedical studies as well as clinical/translational research. While multifactor, dynamic computational analyses improve and become more widespread, the initial step—obtaining relevant raw data from an ever-growing pool of protein biomarkers, single-nucleotide polymorphisms, and other molecular analytes—remains a major rate-limiting operation. Further complicating this process is that data usually are spread over multiple files, and even multiple file types, and thus the task of data aggregation and search becomes both more tedious and vulnerable to error. The process could be expedited via SQL (Structured Query Language), but it would necessitate importing and collating all data sheets into one database, as well as having SQL experience to access and query the data.

### SUMMARY

This specification generally describes a search engine that accepts as input different types of data files and conditions for search parameters, including both single and multiple time points, concatenates those disparate files, and outputs data from the different types of files that satisfies the specified search conditions. The concatenated file can either remain resident in memory or saved to a file, but in either case this allows for searching across disparate data sources and easily generating an output set of results that meet query specifications without first combining all of the data into a single database. The search engine also performs concatenation of a variety of data types and offers automatic quality checks, encryption, and formatting for subsequent machine learning analysis.

In general, one innovative aspect of the subject matter described in this specification can be embodied in methods that include the actions of receiving a selection of a multiple input data files that each include data on which a search is to be performed. The input data files include different types of data files having different data formats. An in-memory data structure is generated based on the data in the input data files. Generating the in-memory data structure includes identifying a data array in at least one of the input data files as a key and aligning the data of the input data files into the data structure based on the key. For each of one or more search parameters, data indicating a condition for the search parameter is received. A set of data that satisfies the condition of each of the one or more search parameters is identified in the in-memory data structure. The set of data is provided as output. Other embodiments of this aspect include corresponding computer systems, apparatus, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination. In some aspects, the data array includes a column or row of a table of the at least one input data file. Identifying the data array can include identifying, as the data array, a common data array that is included in each input data file.

In some aspects, identifying the data array can include receiving data specifying a key file comprising key data array and replacing, in the data structure, a data array corresponding to the key data array with the key data array. Some aspects can include receiving data specifying an output file type. Outputting the set of data can include generating an output file of the output file type and populating the output file with the set of data.

Some aspects include detecting a data format of each input data file. Generating the in-memory data structure can include formatting the in-memory data structure based on the format of each input data file. Formatting the in-memory data structure based on the format of each input data file can include indexing the in-memory data structure by row headers when at least one input data file includes a particular data format and indexing the in-memory data structure by column headers when none of the input data files have the particular data format.

In some aspects, a first input data file of the input data files includes data specifying single-nucleotide polymorphisms (SNPs) for subjects and a second input data file of the input data files includes other data related to the subjects, but does not include any SNPs. Generating the in-memory data structure can include, for each subject aligning data specifying the SNPs for each subject in the first input data file with the other data related to the subject in the second data file. At least one of the conditions for at least one of the one or more search parameters can include data specifying a particular SNP or a particular genotype of a particular SNP. The data specifying the particular SNP can include a name of the particular SNP or a chromosome and position for the SNP.

In some aspects, identifying, in the in-memory data structure, a set of data that satisfies the condition of each of the one or more search parameters includes, for each search parameter, finding the search parameter in the in-memory data structure, identifying a list of data arrays for which data in the data arrays satisfies the condition for the search parameter, and adding the list of data arrays to a cumulative list of data arrays.

In some aspects, receiving, for each of one or more search parameters, data indicating a condition for the search parameter includes populating search parameter entry user interface elements with headers of data arrays of the input data files and receiving a selection of at least one header using the search parameter entry user interface elements.

In some aspects, outputting the set of data can include generating an electronic medical record that includes the set of data. Receiving, for each of one or more search parameters, data indicating a search condition for the search parameter can include receiving one or more patient identifiers. At least one of the input data files can include medical data for patients and at least one of the input data files can include genome data for the patients. Generating the electronic medical record can include generating an electronic medical record that includes medical data and genome data for one or more patients identified by the one or more patient identifiers.

The subject matter described in this specification can be implemented in particular embodiments and may result in one or more of the following advantages. Search engines described in this document can accept as input multiple data files of different file types and having different formats for storing data, generate an in-memory data structure that includes the data of the multiple data files, e.g., by joining or otherwise combining the data files, and perform queries on the in-memory data structure. This can enable different data files to be searched without building large long-term databases to include vast amounts of data, resulting in faster searching, reduced data storage requirements, flexible searching based on user-selected files, and without requiring database experts to build and maintain such large databases. The search engine can identify the types and formats of the data in the input data files, combine the data based on common types of data included in the data, and generate the in-memory model in such a manner that enables the combined data to be searched quickly and efficiently. The in-memory data structure can reside in short-term memory, such as in Random Access Memory (RAM). In this way, the in-memory data can be searched quickly without the latency required to generate output files that include the concatenated data files. This also reduces data storage errors that can occur when generating the output files, e.g., by exceeding data limits of particular file types. The joined data files can also be saved into a single flat file, e.g., in response to a user request.

In some particular implementations, the search engine can read and recognize data files that include genome data, such as single-nucleotide polymorphisms (SNPs) and combine this genome data into an in-memory, RAM-resident data structure that includes other types of data, e.g., data related to subjects that have the SNPs. This enables users to submit queries for genotypes of SNPs, which in turn enables researchers in genomic studies to quickly find patient subsets within a substantial amount of data, without having to generate intractably large databases to store such data. In addition, the search engine can be used to directly concatenate free-form files with a wide variety of data types (e.g., genomic, clinical, singular or multiple time points) into a single flat file that retains relational links to underlying data, and the output files of any operations of the search engine can be automatically encrypted, checked for consistency or missingness, and formatted for downstream machine learning analysis. For example, the concatenated data file of the in-memory data structure can be encrypted upon each cycle of concatenation, thereby potentially forming the basis of a free-form electronic medical record that includes current- and next-generation genomic data. In addition, the output data can be formatted for downstream machine learning analysis based on the data that satisfies the conditions of the search parameters. As an example, if the data are to be analyzed statistically using Two-way Analysis of Variance (ANOVA), each and every one of the selected subjects' multiple time points can be extracted and set as an individual observation, and this new data set can be sorted by the observed time. Other formats of the data can be used if the intended subsequent analysis involves a machine learning algorithm. This can save substantial time in preparing for and performing statistical or machine learning analyses.

The search engine can concatenate free-form and endless combinations of input files into a single flat file, e.g., within the in-memory data structure, that retains relational links to the underlying data of the input files. The input data that are concatenated into the single flat file can includes single time points, multiple time points (e.g., time series data), of a combination thereof. For example, the search engine can concatenate, into a single file, a first input file that includes values for biomarkers of subjects for multiple time points, a second input file that includes demographic data for the subjects, and a third input file that includes genome data for the subjects. In addition, the search engine can concatenate genome-scale data as well as other genomics data directly to demographics, clinical and biomarker data. The search engine can then perform a search on any combination of the above concatenated data without first having to input all of these data into a structured relational database.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an example of an environment in which a search system receive data specifying data files and conditions for search parameters and outputs data of the data files that satisfies the conditions.

FIG. 2 is a user interface for specifying data files and conditions for search parameters.

FIGS. 3A-3C are user interfaces in which data files and conditions for search parameters are specified.

FIG. 4 is a flow diagram of an example process for data specifying data files and conditions for search parameters and outputting data of the data files that satisfies the conditions.

FIG. 5 is a flow diagram of an example process for data specifying data files and conditions for search parameters and outputting data of the data files that satisfies the conditions.

FIG. 6 is a block diagram of a computing system that can be used in connection with computer-implemented methods described in this document.

Like reference numbers and designations in the various drawings indicate like elements.

### DETAILED DESCRIPTION

This specification generally describes a search engine that accepts as input different types of data files and conditions for search parameters, and outputs data from the different types of files that satisfies the conditions. The search engine can combine, e.g., concatenate, the data of the multiple data files into a common in-memory data structure that can be exported into a single flat file, and query the in-memory data structure to identify data that satisfies the conditions of the search parameters (e.g., a subset or portion of the data from the various input data files such as portions of a genome sequence that satisfy the conditions of the search parameters). This can provide a flexible search environment that allows users to query particular files without the need for building a large database that includes the data of the multiple files and without including in the search space unnecessary or unwanted data.

In a particular example, the search engine allows users to select data files that include genome data and data files that include non-genome data, which may be referred to in this document as standard data. Absent the techniques described in this document, such querying of a combination of data included in genome data files and non-genome data files would not be possible without spending large amounts of time and resources manually combining the data into a database.

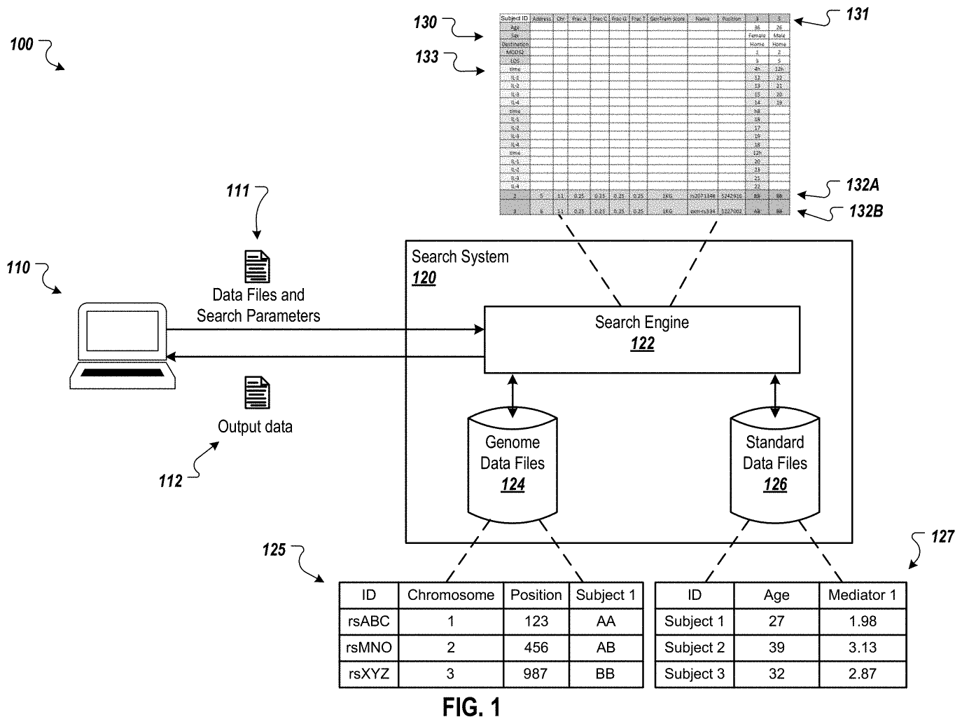

FIG. 1 is an example of an environment 100 in which an example search system 120 receives data specifying data files and conditions for search parameters and outputs data of the data files that satisfies the conditions. FIG. 1 is described largely in terms of data files that include genome data and data files that include non-genome, or standard data. However, the search system 120 can perform the same or similar functions for various types of data files that include different types of data and/or data in different formats or different data structures.

The example search system 120 includes a search engine 122, a set of genome data files 124, and a set of standard data files 126. The search system 120 can be implemented as one or more computers in one or more locations, and the search engine 122 can be hosted on one or more of these computers. The search engine 122 can be a software application running on the one or more computers of the search system 120.

The genome data files 124 and the standard data files 126 can be stored in the same or different data storage locations, e.g., in the same or different hard drives, flash memory, cloud-based (or other network-based) storage, etc. In some implementations, rather than store the files at the search system 120, a user using a user terminal 110, e.g., a personal computer or other computing device, can upload files to be searched to the search system 120. In another example, the search engine 122 can be installed on the user terminal 110 such that the search engine performs searches on the user terminal 110 using files stored locally on the user terminal 110 or elsewhere, e.g., in cloud-based storage.

Each genome data file 124 can include genome data, such as genome data for a set of human subjects, e.g., human patients. As shown in the example table 125, a genome data file 124 can include information such as identifiers of subjects (e.g., Subject 1), identifiers (e.g., unique names) for single-nucleotide polymorphisms (SNPs), the chromosome of each SNP, and the chromosome position of each SNP, and the genotype (e.g., AA, AB, or BB) of the SNP for each subject that has the SNP. The genome data files 124 can further include other appropriate genome data and/or data of subjects for which the genome data is included in the genome files. In some implementations, the genome files include output files generated from Illumina™ Genome Studio. In such files, the subject identifiers are included in column headers as shown in the table 125, e.g., Subject 1 is a column header. In addition, genome data files typically include column (or row) headers for chromosomes and/or positions.

The standard data files 126 can include non-genome data. For example, a standard data file 126 can include other information about a set of subjects for which genome data is included in the genome data files 124. As shown in the example table 127, a standard data file 126 can include identifiers of subjects and, for each subject, the age of the subject and/or other demographic or other appropriate data about the subject, and values of one or more biomarkers (in this example, circulating inflammatory mediators) for each subject.

The search engine 122 is configured to accept as input various types of data files with different data formats or data structures. In this example, the search engine 122 can accept genome data files 124 and standard data files 126 having different formats and different types of data. As shown in the tables 125 and 127, the data for subjects can be in different formats. For example, in the table 125, the subject identifiers are column headers and, in the table 127, the subject identifiers are values in the first cell of each row. The data files can be, for example, in the form of spreadsheets (e.g., Microsoft™ Excel™ files, Structured Query Language [SQL] files, and/or comma-separated values [CSV] files). Some data files can include single time point data, e.g., demographic data, and some data files can include multiple time point data, e.g., a sequence of values for biomarkers measured at different times.

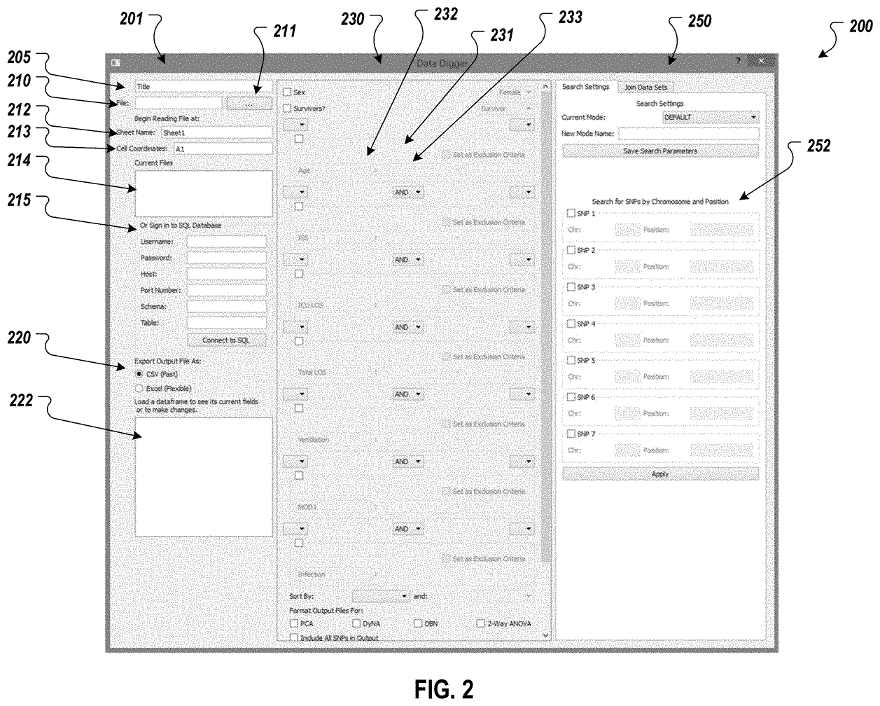

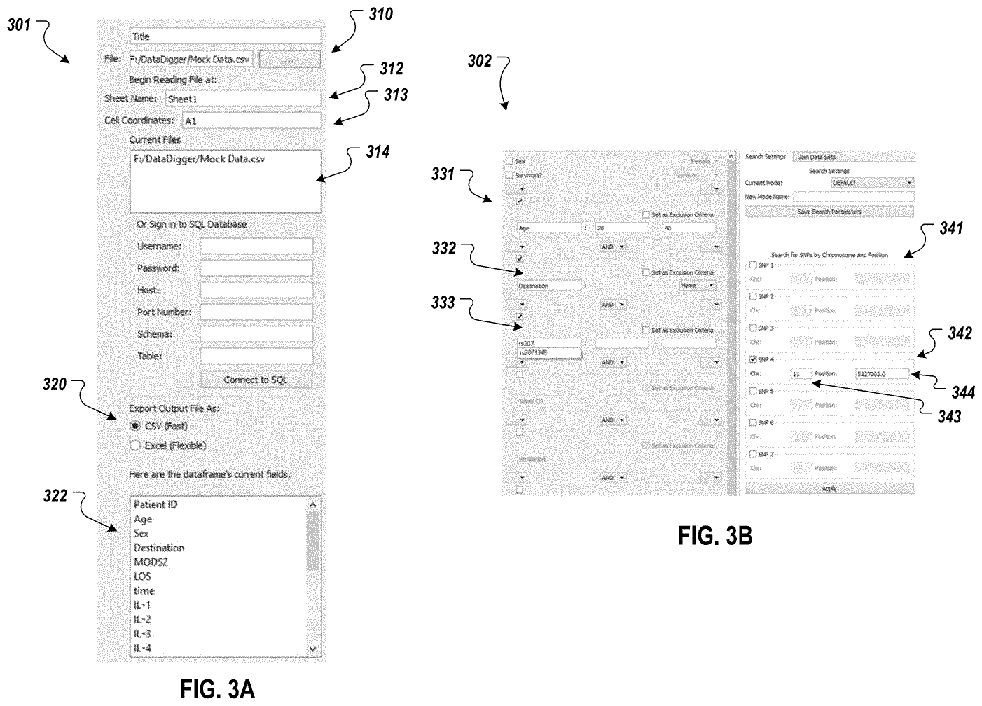

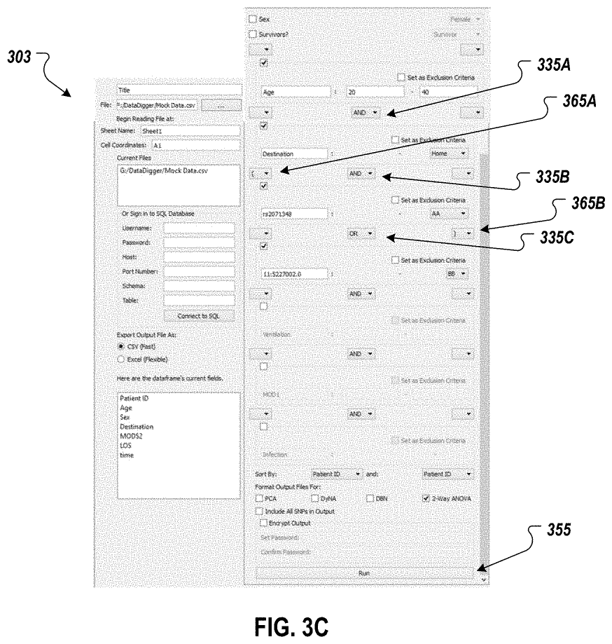

A user can initiate a search for data included in one or more files using a user interface provided by the search engine 122. Example user interfaces provided by the search engine 122 are illustrated in FIGS. 2 and 3A-3C and described below.

In general, a user can use a user interface to select (or otherwise specify) one or more data files and specify conditions for one or more search parameters. The user terminal 111 can then provide data 111 specifying the data file(s) and the condition(s) for the search parameter(s) to the search engine 122. For example, a user may specify one or more genome data files 124 and/or one or more standard data files 126. The user may also specify one or more conditions for genome data (e.g., specify one or more SNPs) and one or more conditions for standard data (e.g., age, sex and/or geographic location of subjects).

The search engine 122 can obtain the specified data file(s), aggregate the data of the data file(s), and generate a data structure 130 that includes the aggregated data. This data structure 130 can be an in-memory data structure stored in memory of the search system 120 (or user terminal 110 if the search engine 122 is implemented locally on the user terminal 111). For example, the data structure 130 can be stored in RAM of the search system 120 or user terminal 110. This enables the search engine 122 to more quickly query the data stored in the data structure 130 relative to databases stored on hard drives, flash memory, or other longer-term data storage devices. The search engine 122 can export the in-memory data structure into a single flat file that can be stored in longer term storage, such as in a hard drive, flash memory, etc. In some implementations, the data structure 130 is a data frame, such as the Pandas DataFrame, which is a two-dimensional labeled data structure with columns that can be of different types.

The search engine 122 can automatically format the data structure 130 based on the detected type of data files and/or the format of data detected in the selected data files. For example, if a single data file or multiple data files having the same data file type and same data format are selected, the search engine 122 can format the data structure 130 to match the data format of the data file(s), e.g., by concatenating the data files together and/or performing a same type of conversion process to reform each data file in the same way as they are aggregated or merged.

If different types of data files are selected, the search engine 122 can format the data structure 130 to include the different types of data included in the data files. The example data structure 130 includes both genome data and standard data from one or more genome data files 124 and one or more standard data files 126. In this example, the data structure 130 includes the subject identifiers as column headers 131 (e.g., subject identifiers 3 and 5) following the column for the chromosome position of the SNPs. The search engine 122 can be configured to generate a data structure 130 having this format when a user selects both a genome data file 124 and a standard data file 126.

In other examples, the search engine 122 can be configured to generate data structures having different formats based on the types of selected data files. For example, the search engine 122 can include a particular data structure format for each possible combination of data files (or combination of data formats) accepted by the search engine 122.

The example data structure 130 includes rows 133 of data for each subject, e.g., aggregated from the selected standard data file(s) and rows 132A and 132B of genome data. In this example, the genome data includes data for each SNP included in the selected genome data file(s) and, for each subject, the genotype of the SNP for that subject. As described in more detail below, the search engine 122 can aggregate and combine the data based on common types of data arrays (e.g., rows or columns) in the selected data files. In this example, the search engine 122 combined the genotype for each subject with the appropriate subject based on the subject identifier column headers in the selected genome data file(s) and the subject identifier column in the selected standard data file(s).

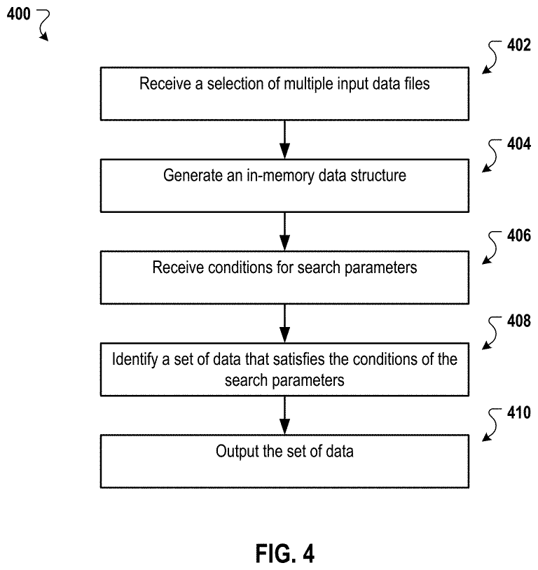

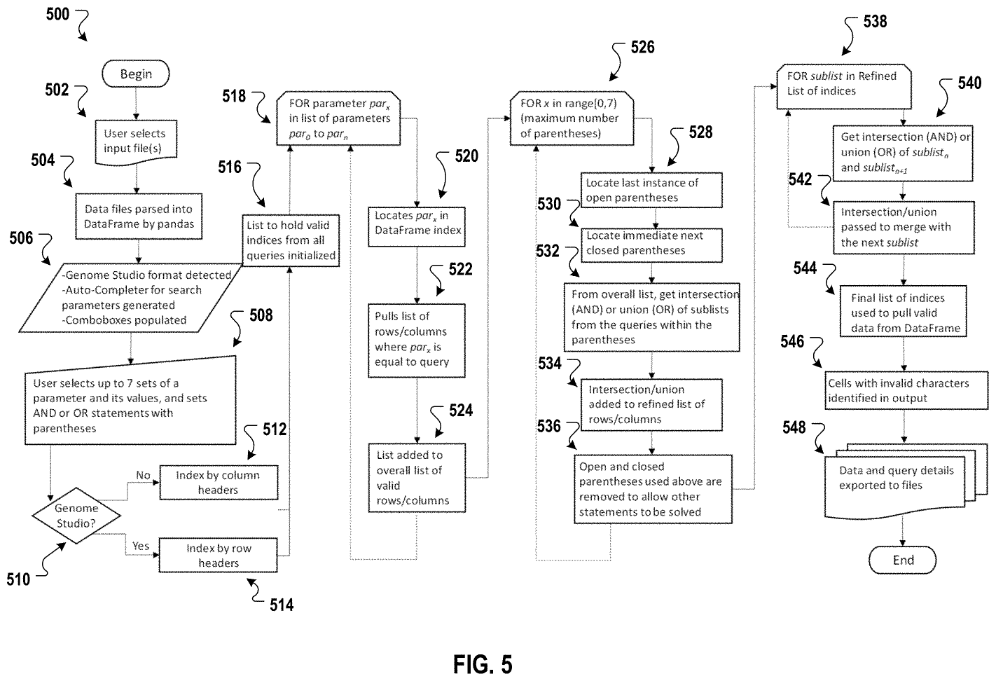

After generating the data structure 130, the search engine 122 can query the data in the data structure 130 based on the specified conditions for the search parameters. Example processes for querying the data of a data structure are illustrated in FIGS. 4 and 5 and described below. The search engine 122 can output the data that satisfy the conditions as output data 112. The output data 112 can be included in a data file, e.g., in a type of data file selected by the user using a user interface of the search engine 122. For example, the user interface can provide several output data file options from which the user can select.

In some implementations, the search engine 122 can enable the user to save search parameters, e.g., including the specified data file(s) and/or the conditions for the search parameters. In this way, the user can repeat the same search using the same or different data files at a later time.

In some implementations, the search engine 122 can encrypt the output data file, e.g., if requested by the user. As the output data files can include sensitive information about subjects, e.g., patients, the encryption protects the data if obtained by other parties. In one example, the search engine 122 can encrypt the output data 112 using a 256-bit Advanced Encryption Standard (AES) encryption algorithm prior to transmitting the output data 112 to the user terminal 110. These encrypted data files can be stored, e.g., as medical records, that can include current-generation genome data and next-generation genome data that can be studied, e.g., using machine learning techniques.

For example, a user of the search engine 122 can use the search engine 122 to generate electronic medical records for one or more patients by selecting input data files that include medical information for the patient(s) and/or genome data for the patient(s). To generate a medical record for one or more patients, the user can specify, as part of the conditions for the search parameters, an identifier for each patient and conditions for any parameters that the user wants included in the electronic medical records. The search engine 122 can identify, in the in-memory data structure and for each patient identifier, medical and/or genome data for the patient identified by the patient identifier and include this data in the output data.

To preserve the privacy of these electronic medical records, the search engine 122 can encrypt the electronic medical records, e.g., by encrypting the file that includes the medical records. A user, e.g., a researcher with the appropriate decryption key, can then search these medical records to find, for example, information about current-generation genome data and next-generation genome data. Such data for multiple patients can also be provided as input to machine learning models.

The search engine 122 can also format the output data for downstream machine learning analysis, which can save substantial time in performing the machine learning analysis. For example, if the data are to be analyzed statistically using Two-way Analysis of Variance (ANOVA), each and every one of the selected subjects' multiple time points can be extracted and set as an individual observation, and this new data set can be sorted by the observed time. Other formats of the data can be used if the intended subsequent analysis involves a machine learning algorithm, e.g., based on the machine learning algorithm being used.

FIG. 2 is a user interface 200 for specifying data files and conditions for search parameters. The example user interface 200 includes an input/output settings area 201, a main search area 230, and a search settings area 250. The input/output settings area 201 enables a user to select one or more input data files and optionally portions of the input data file(s) (to the exclusion of other portions of the input data file(s)) for a search. The input/output settings area 201 also enables a user to select the type of output file for the output data that satisfies the search conditions.

The input/output settings area 201 includes a title element 205 that enables a user to input a title for the search. If the user saves the search settings, the search engine 122 can save the search settings using the title, e.g., as the title for the search settings. The input/output settings area 201 also includes an input file selection element 210 that includes a file selector button 211 that enables the user to browse a file system (e.g., a file system of the user terminal 110 or the search system 120) for each input data file for the search.

The input/output settings area 201 also includes a sheet name element 212 and a call coordinates element 213 that enables the user to select portions of a data file for which data should be included in the data structure that will be searched. The sheet name element 212 enables the user to select particular sheets of a spreadsheet and the cell coordinates element 213 enables the user to select particular cells of the spreadsheet. For example, the sheet name element 212 and the call coordinates element 213 can enable the user to select from where in the input data file the search engine 122 should start reading data. This can be particularly useful if some data should be excluded and/or if some rows of a spreadsheet include information about the data in the spreadsheet or instructions for users of the spreadsheet that is not part of the actual data of the spreadsheet. If the user selects particular sheets and/or particular cells of particular sheets, the search engine 122 will ignore the data included in non-selected sheets and/or cells and not include that data in the data structure that will be searched.

The input/output settings area 201 also includes a current files window 214 that shows a list of the input data files that have already been selected by the user. In this example, no input data files have yet been selected and added for the search. The input/output settings area 201 also includes an SQL selection element that enables the user to log into an SQL database so that the search engine 122 can extract data from a protected SQL database.

The input/output settings area 201 also includes an export file type element 220 that enables the user to select the output file type for the output data that satisfies the search conditions. In this example, the user can select from Excel™ or CSV output data file types.

The input/output settings area 201 also includes current fields window 222 that shows the fields of the input data files or portions thereof that will be included in the data structure that will be searched. This enables the user to view which fields can be queried by specifying conditions for the fields. For example, if an input data file includes subject identifiers and age, the current fields window 222 would show “subject identifiers” and “age” as current fields. As more input data files are selected, additional fields may be shown in the current fields window 222 corresponding to fields detected in the additional input data files.

The main search area 230 enables the user to specify the search conditions, e.g., the conditions of the parameters that will be used by the search engine 122 to search a data structure generated using the data of the selected input data files that were selected in the input/output settings area 201. The main search area 230 includes multiple search parameter elements 231. Each search parameter element 231 enables the user to specify a condition for a search parameter. Each search parameter element 231 includes a field element 232 that enables the user to specify a field of the input data files to which the condition will apply. For example, the first search parameter element 231 is an “age” field. Each search parameter element 231 also includes a value element 233 that enables the user to enter the condition, e.g., a value or range of values. For example, the value element 233 for the “age” field may be a range of ages between 21 and 35. In this example, unless the exclusion checkbox is selected, the search engine 122 would only search the input data for, and output data for, subjects within the specified age range in the value element 233. If the exclusion checkbox is selected, the search engine 122 would only output data for subjects outside of that specified age range.

In some implementations, the search engine 122 populates drop-down menus (or other search parameter entry user interface elements) of the field elements 232 with the headers (e.g., row or column headers) of the input data files. This makes it easier for a user to select the search parameters using the field elements. For example, a user can generate a condition for a search parameter by simply selecting a search parameter from the drop-down menu and specifying the condition for the search parameter.

The search settings area 250 enables the user to specify search settings and optionally specify SNPs based on chromosome and position. For example, the search settings area 250 includes multiple SNP elements 252 that enable the user to specify SNPs based on chromosome and position. Alternatively, the user can specify the SNPs by identifier using the search parameter elements 231 of the main search area 230.

FIGS. 3A-3C are user interfaces 301-303 in which data files and conditions for search parameters are specified. A user can use the user interfaces 301-303 to select input data files and specify search conditions and initiate a search of the input data files based on the search conditions. The user interfaces 301-303 illustrate a sequence in which a user selects the input data files and specifies the search conditions using portions of the user interface 200 of FIG. 2

Referring to FIG. 3A, the user interface 301 shows an input/output settings area, e.g., similar to the input/output settings area 201 of FIG. 2. In this example, the user has selected a file “Mock Data.csv” using a file selector element 310. The user has also selected to start reading the data from this input data file at cell coordinate “A1” of “Sheet 1” using a sheet name element 312 and a cell coordinates element 313. If “Sheet1” is the first sheet in the csv file, then all of the data of the csv file would be included in the data structure that will be searched. If the user had selected a second sheet, e.g., “Sheet 2,” the search engine 122 would skip “Sheet 1,” and only use data starting at “Sheet 2,” in this example. After selecting the “Mock Data.csv” file, this input data file is shown in the current files window 314.

The user has also selected that the output data file should be a csv file using an export file type element 320. A current fields window 322 shows the fields of the input data files selected thus far, e.g., the fields of the “Mock Data.csv” file. For example, the fields include Patient ID, age, sex, etc. The search engine 122 can evaluate the input data files, identify array headers, e.g., column and/or row headers, for data in the data files, and populate the current fields window 322 as the user selects input data files, e.g., after each file is selected and without waiting for all input data files to be selected. This enables the user to view the fields for which search conditions can be specified, e.g., the fields of the input data files that can be queried.

Referring to FIG. 3B, the user interface 302 shows a main search area, e.g., similar to the main search area 230 of FIG. 2. In this example, the user has specified several search conditions. In search parameter element 331, the user has specified a search parameter “Age,” based on the field “Age” of the input data file(s). The user has also specified a condition for this search parameter, e.g., an age range of 20-40. In this example, the output data would only include data for subjects having an age within this age range.

Similarly, the user has specified a search parameter “Destination” based on the field “Destination” and a condition that the value of this field must be “Home” using search parameter element 332. In addition, the user is specifying an SNP search parameter “rs2071348.” In this example, the user has entered “rs207” and the search engine 122 is providing an autocomplete suggestion of “rs2071348” based on this SNP being included in one of the input data files.

Rather than type the name (or other identifier) of an SNP into a search parameter element 333, the user can alternatively use an SNP element 342 of an SNP selector area 341 to specify SNPs based on chromosome and chromosome position. In this example, the user has selected an SNP having chromosome 11 using a chromosome selector element 343 and has selected position 5227002.0 using a position selector element 344.

Referring to FIG. 3C, a user interface 303 shows a combination of the input/output settings area and the main search area after the user has selected the input data file and specified the search conditions. In this example, the user has specified the genotype AA for the SNP “rs2071348” and the genotype BB for the SNP corresponding to chromosome 11 at position 5227002.0.

The main search area includes parenthesis elements, e.g., parenthesis elements 365A and 365B, and logical operator elements, e.g., logical operator elements 335A-335C. An AND operator enables users to search for the intersections of search conditions and the OR operator enables users to search for unions between search conditions.

In this example, the user has also selected parenthesis to enclose the two SNPs using parenthesis elements 365A and 365B and has selected an OR operator between the two SNPs using a logical operator element 335C. In addition, the user has selected AND operators (or they are provided as defaults) using logical operator elements 335A and 335B. The final search query would be data for subjects having an age between 20-40 with a destination of home, and this data would include the genotype for each of the two SNPs for each subject matching the age and destination criteria, and optionally additional data for the two SNPs.

FIG. 4 is a flow diagram of an example process 400 for receiving data specifying data files and conditions for search parameters and outputting data of the data files that satisfies the conditions. The example process 400 can be performed by the search system 120 of FIG. 1. Operations of the process 400 can also be implemented as instructions stored on non-transitory computer readable media, and execution of the instructions by one or more data processing apparatus can cause the one or more data processing apparatus to perform the operations of the process 400. For brevity, the process will be described in terms of the search engine 122 running on the search system 120 of FIG. 1.

The search engine 122 receives a selection of multiple input data files (402). A user can select the input data files using one of the user interfaces described above. The input data files can include different types of data files. For example, the input data files can include one or more genome data files that include genome data, e.g., data for SNPs, and one or more non-genome data files that do not include genome data. The different types of data files can be formatted differently and/or include different data structures. For example, as shown in the tables 125 and 127 of FIG. 1, the subject identifiers can be column headers in genome data files and can be values of cells in each row of standard (non-genome) data files.

Other types of data, e.g., data that is not related to genomes such as machine learning data, geographic map data, etc. can also be included in different types of files with different data formats. For example, data files with Global Positioning System (GPS) data may be formatted differently from data files that include canvases for maps). The search engine 122 can perform similar functions for such data files.

The search engine 122 generates an in-memory data structure based on the input data files (404). As described above, the search engine 122 can format the in-memory data structure based on the types of data files selected and/or the format of the data in the input data files. For example, the search engine 122 can detect the type of data based on headers for arrays included in the data files and/or the format of the data files. In a particular example, the search engine 122 can determine that a data file includes genome data if there are headers for chromosomes (e.g., a header is “chr” or “chromosome”) and position (e.g., a header is “position”). In another example, the search engine 122 can determine that an input data file includes genome data if the data file includes patient identifiers in column headers.

The in-memory data structure can be a data frame, e.g., a Pandas DataFrame. The in-memory data structure arranges the data on the input data files in a common format, e.g., within the data frame.

As described in more detail below with reference to FIG. 5, the search engine 122 can index the in-memory data structure based on the types of input data files selected and/or their data formats. For example, the search engine 122 can index the data in the data structure by row headers if the input data files include genome data. If not, the search engine 122 can index the data in the data structure by column headers, e.g., if the standard data files are indexed by column headers.

Generating the in-memory data structure includes aligning and aggregating the data of the input data files and populating the in-memory data structure with the aggregated data. As multiple input data files can include data for a same entity, e.g., a same subject, the search engine 122 can find a common data array that is common to two or more input data files. This common data array can serve as a key for aligning the data of the two or more input data files. For example, the search engine 122 can find that two or more data files include data arrays for subject identifiers. A genome data file can include column headers that include subject identifiers and a standard data file can include a row for each subject identifier. In this example, the search engine 122 can identify a common subject identifier in two or more input data files and aggregate the data for that subject identifier in the in-memory data structure. In this way, the in-memory data structure includes an array (e.g., row or column) that includes the combined data of the multiple data files that include data for each subject identifier.

The search engine 122 can perform similar operations for each common type of data array (e.g., each pair of data arrays having the same type of data for overlapping entities). For example, if multiple genome files include different data for the same SNPs, the search engine can aggregate the data for each SNP in the in-memory data structure in a similar manner.

In some implementations, the search engine 122 can replace the data of a data array with data of a key data array. For example, if the subjects are patients, it may be preferable to not include patient identifiers in the output data. In this example, the search engine 122 can receive data specifying a key file that includes a key data array. The key data array can include generic subject identifiers for replacing the actual subject identifiers. For example, the key data file can include a first data array with actual subject identifiers and a second data array with generic subject identifiers such that the actual subject identifiers are mapped to generic subject identifiers. In a particular example, both data arrays can be columns that are side by side. Each row can map an actual subject identifier to a generic subject identifier.

The key data array can include the same header, e.g., “Patient ID” or “Subject ID,” as the header for the data array that should be replaced. Prior to generating the in-memory data structure, the search engine 122 can identify, in the input data files, any data arrays that have this header and replace the data in the data arrays with the data of the key data array.

The search engine 122 receives data indicating a respective condition for each of one or more search parameters (406). For example, a user can use one of the user interfaces described above to input the conditions for the search parameters. In implementations in which genome data is being searched, the search parameters can include genome search parameters, e.g., particular SNPs and/or particular genotypes for particular SNPs. In this way, a user can search for subjects that have the particular SNPs and/or the particular genotypes of the particular SNPs. The search parameters can also include non-genome search parameters, such as age or other data about the subjects. In this way, the user can limit the output data to particular subsets of the subjects having the particular SNPs and/or the particular genotypes of the particular SNPs.

The search engine 122 identifies a set of data that satisfies the conditions of the search parameters (408). In general, the search engine 122 can query the in-memory data structure to identify data, if any, that satisfies each search condition. This querying can include, for each search parameter, finding the data array(s) for the search parameter in the in-memory data structure, identifying a list of data arrays for which data in the data arrays satisfies the condition for the search parameter, and adding the list of data arrays to a cumulative list of data arrays that are determined to satisfy the condition of the search parameter. After the cumulative list of data arrays is generated by processing each condition, the search engine 122 can generate a set of output data. The cumulative list of data arrays can be adjusted by adding or removing certain data arrays according to the search parameter conditions and search logic (e.g., logical operators, parentheses) specified in the search query. An example technique for generating the output data is described in more detail with reference to FIG. 5.

The search engine 122 outputs the set of data (410). Prior to outputting the set of data, the search engine 122 can format the data based on the output data file selected by the user and generate an output data file that includes the formatted data. As described above, the search engine 122 can encrypt the output data file and/or format the output data for downstream machine learning analysis. The search engine 122 can then transmit the output data file to a user terminal and/or present the output data in a user interface at the user terminal.

FIG. 5 is a flow diagram of an example process 500 for receiving data specifying data files and conditions for search parameters and outputting data of the data files that satisfies the conditions. In some implementations, the example process 400 can be performed by the search system 120 of FIG. 1. Operations of the process 400 can also be implemented as instructions stored on non-transitory computer readable media, and execution of the instructions by one or more data processing apparatus can cause the one or more data processing apparatus to perform the operations of the process 400. For brevity, the process will be described in terms of the search engine 122 running on the search system 120 of FIG. 1.

The search engine 122 receives a selection of input data files (502). For example, a user can select input data files using one of the user interfaces described above. If any of the data files are encrypted, the search engine 122 can prompt the user for a password for the input data file.

The search engine 122 parses the data files and generates an in-memory data structure (504). The in-memory data structure can be a Pandas DataFrame. As described above, the input data files can include genome data and/or non-genome data.

In some implementations, the search engine 122 can check the in-memory data structure for genome data. For example, the search engine 122 can determine whether in-memory data structure includes chromosome or position headers and, if so, determine that the in-memory data structure includes genome data.

The search engine 122 can also check the in-memory data structure for data listed across multiple time points (multiplex) and data listed once (singleton). The search engine 122 can split the in-memory data structure, e.g., data frame, into multiplex and singleton sections. In this example, genome data can be treated as singleton data.

The search engine 122 can add the multiplex data frames to a list, whereas singletons are appended into one large data frame. These data frames can be referred to as initial data frames. The search engine 122 can determine the maximum number of time points across all input data files by the maximum repetitions of the time point header across all multiplex sections.

If any genome data are detected in any of these initial data frames (e.g., based on header names indicative of genome data), the search engine 122 can transpose any data listed in a simple format to fit a genome format, e.g., as shown in the data structure 130 of FIG. 1. The search engine 122 can combine the parameters from all multiplex data frames into a single data frame. This can be repeated to match the maximum number of time points. The search engine 122 can then convert the maximum number of time points into a single series which will hold the row headers for this data frame.

For each subject, the search engine 122 can format the data for each point of time for the subject in the data frame. This can include organizing the data for the time point to match the combined list of parameters. Any missing data, e.g., for a given time point, can be filled be a default value, such as “NaN” to that its length matches that of the parameter list. The search engine 122 can then add the organized data for each time point for each subject to a cumulative series of the subject's data.

If genome data are present, the search engine 122 can pop the genome data from the singleton data to be appended at the bottom of the resulting data frame, similar to how the genome data is arranged in the data structure 130 of FIG. 1. The search engine 122 can combine the singleton, multiplex, and (if applicable) the genome data into a single data frame similar to the data structure 130 of FIG. 1. The data structure 130 of FIG. 1 includes demographic and time series data for a set of subjects and genome data in the bottom rows. The search engine 120 can also export the data frame to a CSV or Excel™ file.

The search engine 120 can update the user interface based on the generated in-memory data structure (506). This can include generating autocomplete data for suggesting auto-completions of text entry boxes based on the values of the fields in the in-memory data structure and populating combo-boxes (e.g., drop down menus) with search parameters that can be selected by the user for creating search conditions.

A user specifies conditions for one or more search parameters (508). In this example, the user may be given the option to specify up to seven conditions. In other examples, more or fewer conditions may be allowed. The user can also arrange parentheses around groups of search parameters and specify logical operators (e.g., AND for intersections and OR for unions) between search parameters. The search engine 122 can receive data specifying these selections from the user interface.

The search engine 122 determines whether the in-memory data structure includes genome data (510). In some implementations, the search engine 122 can attempt to locate a column (or row) header for chromosomes. For example, the search engine 122 can attempt to locate a header with “Chr”, “Chromosome”, and/or “Position”. If the search engine 122 determines that the in-memory data structure does not include genome data, e.g., there are no chromosome headers in the in-memory data structure, the search engine 122 uses the column headers as an index for the in-memory data structure (512). If the search engine 122 determines that the in-memory data structure does include genome data, e.g., there are one or more chromosome headers in the in-memory data structure, the search engine 122 uses row headers as an index for the in-memory data structure (514). This search engine 122 uses the selected headers as an index by using those headers to find the parameter names based on the search parameters for which conditions have been specified by the user.

The search engine 122 initializes a list to store valid indices for all of the search parameters (516). For each search parameter, the search engine 122 locates the search parameter in the index for the in-memory data structure (520). The search engine 122 then pulls a list of the data arrays (e.g., columns and/or rows) where the value of the search parameter satisfies the condition set of the search parameter by the user. The search engine 122 adds the identifier data arrays to an overall list of valid data arrays. The search engine 122 performs operations 520-524 for each search parameter to build the overall list of valid data arrays.

The search engine 122 can perform operations 526-536 to handle parenthesis specified by the user. For this discussion, assume that the generated search query is Sex==Male AND (Blood Pressure <90 OR Reperfusion==True OR Base Deficit/Excess <5).

The search engine 122 identifies a maximum number of potential parentheses and initializes a count from 0 to the maximum number to process the parentheses in order (526). In this example, the range is from 0 to 7. For each count, the search engine 122 locates a last instance of open parentheses (528) and locates the next immediate next closed parentheses (530). From the overall list of valid data arrays, the search engine obtains the intersection (AND) or the union (OR) of sublists from the search parameters within the parentheses. In the example provided above, the search engine would identify the union of data arrays for subjects that have a blood pressure <90, a reperfusion, or a base deficit/excess <5.

The search engine 122 adds the intersection or union within the current parentheses to a refined list of valid data arrays (534). In this example, the search engine 122 would add the data arrays for the union of subjects that that have a blood pressure <90, a reperfusion, or a base deficit/excess <5 to the refined list of data arrays.

The search engine 122 then removes the open and closed parentheses used in the current iteration from the overall search query to allow the remaining search statements to be solved (536). The search engine 122 can repeat this process using operations 526-536 for each set of parentheses to generate the refined list of indices. In this example, the refined list of indices would include a sublist of indices for males and a sublist of indices for subjects that have a blood pressure <90, a reperfusion, or a base deficit/excess <5.

For each sublist in the refined list of indices (538), the search engine 122 determines the intersection or union of the sublist and its immediately following sublist based on the logical operator between these two sublists (540). Continuing the previous example, the search engine would determine the intersection of the sublist of males and the sublist of subjects that have a blood pressure <90, a reperfusion, or a base deficit/excess <5 as the user specified an AND operator between the males search condition and the parenthetical search condition.

The sublist resulting from operation 540 is passed back to merge with the next sublist, if any (542). In this way, the search engine 122 can process each pair of sublists in order since the parenthesis were previously handled.

The search engine 122 obtains a final list of indices for data arrays that satisfy the search conditions after processing all of the sublists in the refined list of indices. The search engine 122 can then obtain the data from each data array indexed by the final list of indices (544).

The search engine 122 can identify cells with invalid characters (546). The search engine 122 can ignore or remove such characters.

The search engine 122 can then output the data and optionally the search query details (548). For example, the search engine 122 can generate an output file of the type selected by the user, format the data, and populate the data file with the formatted data. The search engine 122 can then provide the output data file to the user, e.g., to the user terminal of the user or present the data in a user interface of the user terminal.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML, page, to a user device, e.g., for purposes of displaying data to and receiving user input from a user interacting with the user device, which acts as a client. Data generated at the user device, e.g., a result of the user interaction, can be received from the user device at the server.

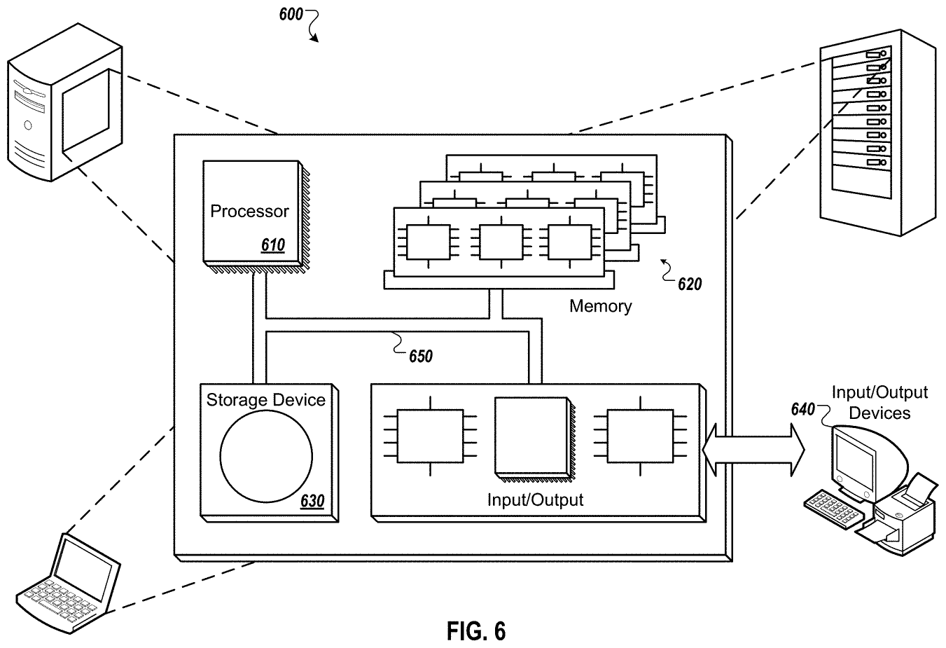

An example of one such type of computer is shown in FIG. 6, which shows a schematic diagram of a generic computer system 600. The system 600 can be used for the operations described in association with any of the computer-implemented methods described previously, according to one implementation. The system 600 includes a processor 610, a memory 620, a storage device 630, and an input/output device 640. Each of the components 610, 620, 630, and 640 are interconnected using a system bus 650. The processor 610 is capable of processing instructions for execution within the system 600. In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 may be a floppy disk device, a hard disk device, an optical disk device, or a tape device.

The input/output device 640 provides input/output operations for the system 600. In one implementation, the input/output device 640 includes a keyboard and/or pointing device. In another implementation, the input/output device 640 includes a display unit for displaying graphical user interfaces.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus, the method comprising:
receiving a selection of a multiple input data files that each include data on which a search is to be performed, wherein the input data files include different types of data files having different data formats;
generating, based on the data in the input data files, an in-memory data structure that includes the data of the input data files arranged in a common format, wherein generating the in-memory data structure includes identifying a data array in at least one of the input data files as a key and aligning the data of the input data files into the data structure based on the key;
receiving, for each of one or more search parameters, data indicating a condition for the search parameter;
identifying, in the in-memory data structure, a set of data that satisfies the condition of each of the one or more search parameters; and
outputting the set of data.

2. The method of claim 1, wherein the data array comprises a column or row of a table of the at least one input data file.

3. The method of claim 1, wherein identifying the data array comprises identifying, as the data array, a common data array that is included in each input data file.

4. The method of claim 1, wherein identifying the data array comprises:
receiving data specifying a key file comprising key data array;
replacing, in the data structure, a data array corresponding to the key data array with the key data array;

5. The method of claim 1, further comprising receiving data specifying an output file type, wherein outputting the set of data comprising generating an output file of the output file type and populating the output file with the set of data.

6. The method of claim 1, further comprising detecting a data format of each input data file, wherein generating the in-memory data structure comprises formatting the in-memory data structure based on the format of each input data file.

7. The method of claim 6, wherein formatting the in-memory data structure based on the format of each input data file comprises indexing the in-memory data structure by row headers when at least one input data file comprises a particular data format and indexing the in-memory data structure by column headers when none of the input data files have the particular data format.

8. The method of claim 1, wherein:
a first input data file of the input data files comprises data specifying single-nucleotide polymorphisms (SNPs) for subjects and a second input data file of the input data files includes other data related to the subjects, but does not include any SNPs; and
generating the in-memory data structure comprises, for each subject aligning data specifying the SNPs for each subject in the first input data file with the other data related to the subject in the second data file.

9. The method of claim 8, wherein at least one of the conditions for at least one of the one or more search parameters comprises data specifying a particular SNP or a particular genotype of a particular SNP.

10. The method of claim 9, wherein the data specifying the particular SNP comprises a name of the particular SNP or a chromosome and position for the SNP.

11. The method of claim 1, wherein identifying, in the in-memory data structure, a set of data that satisfies the condition of each of the one or more search parameters comprises:
for each search parameter:
finding the search parameter in the in-memory data structure;
identifying a list of data arrays for which data in the data arrays satisfies the condition for the search parameter; and
adding the list of data arrays to a cumulative list of data arrays.

12. The method of claim 1, wherein receiving, for each of one or more search parameters, data indicating a condition for the search parameter comprises:
populating search parameter entry user interface elements with headers of data arrays of the input data files; and
receiving a selection of at least one header using the search parameter entry user interface elements.

13. The method of claim 1, wherein outputting the set of data comprises generating an electronic medical record that includes the set of data.

14. The method of claim 13, wherein:
receiving, for each of one or more search parameters, data indicating a search condition for the search parameter comprises receiving one or more patient identifiers;
at least one of the input data files comprises medical data for patients and at least one of the input data files comprises genome data for the patients; and
generating the electronic medical record comprises generating an electronic medical record that includes medical data and genome data for one or more patients identified by the one or more patient identifiers.

15. A computer-implemented system, comprising:
one or more computers; and
one or more computer memory devices interoperably coupled with the one or more computers and having tangible, non-transitory, machine-readable media storing one or more instructions that, when executed by the one or more computers, perform operations comprising:
receiving a selection of a multiple input data files that each include data on which a search is to be performed, wherein the input data files include different types of data files having different data formats;
generating, based on the data in the input data files, an in-memory data structure that includes the data of the input data files arranged in a common format, wherein generating the in-memory data structure includes identifying a data array in at least one of the input data files as a key and aligning the data of the input data files into the data structure based on the key;
receiving, for each of one or more search parameters, data indicating a condition for the search parameter;
identifying, in the in-memory data structure, a set of data that satisfies the condition of each of the one or more search parameters; and
outputting the set of data.

16. The computer-implemented system of claim 15, wherein the data array comprises a column or row of a table of the at least one input data file.

17. The computer-implemented system of claim 15, wherein identifying the data array comprises identifying, as the data array, a common data array that is included in each input data file.

18. The computer-implemented system of claim 15, wherein identifying the data array comprises:
receiving data specifying a key file comprising key data array;
replacing, in the data structure, a data array corresponding to the key data array with the key data array;

19. The computer-implemented system of claim 15, wherein the operations comprise receiving data specifying an output file type, wherein outputting the set of data comprising generating an output file of the output file type and populating the output file with the set of data.

20. A non-transitory, computer-readable medium storing one or more instructions executable by a computer system to perform operations comprising:
receiving a selection of a multiple input data files that each include data on which a search is to be performed, wherein the input data files include different types of data files having different data formats;
generating, based on the data in the input data files, an in-memory data structure that includes the data of the input data files arranged in a common format, wherein generating the in-memory data structure includes identifying a data array in at least one of the input data files as a key and aligning the data of the input data files into the data structure based on the key;
receiving, for each of one or more search parameters, data indicating a condition for the search parameter;
identifying, in the in-memory data structure, a set of data that satisfies the condition of each of the one or more search parameters; and
outputting the set of data.
